(19) United States
(12) Patent Application Publication
(10) Pub. No.: US 2007/0258958 A1
(43) Pub. Date: Nov. 8, 2007

(54) Interactive wound cover

(75) Inventors: Deepa Ghosh, Navi Mumbai (IN); Pushpa Kuchroo, Navi Mumbai (IN); Sudheer Shenoy, Navi Mumbai (IN); Vidhya Rangaswamy, Navi Mumbai (IN); Sonti Venkata Rama Krishna, Navi Mumbai (IN)

Correspondence Address: FOLEY AND LARDNER LLP;SUITE 500, 3000 K STREET NW, WASHINGTON, DC, 20007, US

(73) Assignee: Reliance Life Sciences Private Limited

(21) Appl. No.: 11/705,032

(22) Filed: Feb. 12, 2007

(30) Foreign Application Priority Data
Feb. 14, 2006 (IN) 205/MUM/2006

(51) Int. Cl. A61K 45/00

(52) U.S. Cl. 424/93.7

(57) ABSTRACT

The present invention relates to an interactive wound cover wherein a cultured monolayer of keratinocytes is delivered using a biopolymer. The present invention describes the composition, method of preparation and its properties relating to safety, and efficacy. The wound cover of the present invention is useful in the treatment of wounds and in skin tissue regeneration.

### CROSS-REFERENCES TO RELATED APPLICATIONS

This application claims benefit of the filing date under 35 U.S.C. § 119(e) to provisional Indian Application No. 205/MUM/2006, filed on Feb. 14, 2006, which is entirely incorporated by reference herein.

### FIELD OF THE INVENTION

The present invention relates to an interactive wound cover, its composition and method of preparation. The present invention particularly relates to the delivery of a cultured monolayer of keratinocytes on polymer for skin regeneration.

### BACKGROUND OF THE INVENTION

The Structure of the Skin

Skin is a bilayer organ comprising an outer, thinner, epidermis and an inner, thicker, dermis, both of which have specific properties.

The epidermis is composed mainly of epithelial cells. Keratinocytes are the outermost cells of the epidermis and produce the protein keratin. Keratinocytes are produced from a layer of basal epidermal cells that are anchored to the basement membrane by adhesion molecules, namely fibronectin. These immature basal cells are constantly dividing and migrating towards the surface, to replace lost surface cells, e.g., after an injury. As the cells mature and migrate to the surface they differentiate into keratinocytes and produce keratin, which becomes an effective barrier to environmental hazards such as infection and to excess water evaporation. Replacement of the epidermal layer by this regenerative process takes 2-3 weeks. Cues and biologic stimuli are necessary to direct the proper orientation and mitotic response of the epidermal cells.

The dermis is a dynamic layer of thick connective tissue, in constant turnover, which is divided into a thin superficial layer known as the papillary dermis and the thicker, deeper, portion known as the reticular dermis. The papillary dermis is the major factory for the proteins providing direction for epidermal replication. The papillary dermis also contains the highest blood flow. The primary cell type of the dermis is the fibroblast that produces the key structural extra cellular matrix proteins (ECM) collagen and elastin, as well as matrix or ground substance. In addition, fibroblasts produce the key adhesion molecules necessary for attachment of epidermal cells to the basement membrane, epidermal cell migration and replication. Fibronectin is a key fibroblast derived signal protein for orchestration of healing. The dermis provides durability and flexibility to the skin. It serves as a factory for all components required for replication and repair of epidermis and dermis. It also provides scaffolding for cell migration and conduit for nutrient delivery.

The interface between the epidermis and dermis layers, the dermo-epidermal junction, is a basement membrane rich in the adhesive protein fibronectin that anchors the epidermal cells from above and dermis from below.

Keratinocyte Development

Keratinocyte stem cells reside in the basal layer of the epidermis. Keratinocyte stem cells have a low rate of mitosis and give rise to a population of transient amplifying cells (TA cells). TA cells go through a limited number of divisions, differentiate, and move up in the epidermis. The cells above the basal layer are known as the spinous layer. Under routine microscopy, small bridges resembling spines can be seen between the keratinocytes, which represent intercellular adhesion complexes known as desmosomes. As the cells further differentiate, they synthesize keratohyaline granules, a prominent feature of cells in the granular layer. Proteins synthesized in the granular layer are important in the final stages of epidermal differentiation and include profilagrin, loricrin, involucrin, and cornifin. These molecules are important in the formation of the stratum corneum, the outer most layer of the epidermis, which is composed of dead, cornified keratinocytes.

The major proteins produced by keratinocytes are keratins. Keratins are intermediate filament proteins that form the cytoskeleton of keratinocytes. Keratins are alpha-helical molecules and belong to two families: Type I (acidic keratins) and Type II (basic keratins). During keratin assembly, an acidic and basic keratin pair to form obligate heteropolymers, which are then assembled into filaments. During epithelial differentiation the expression of keratins changes.

Basal cells express keratins 5 and 14. As keratinocytes leave the basal layer, they become larger and synthesize keratins 1 and 10. Different keratins are associated with hair and nail formation. In hyperprolific epidermis, such as psoriasis and atopic dermatitis, keratin 6 and 16 predominate. A congenital blistering disease, epidermolysis bullosa simplex, is due to defects in keratins 5 and 14 resulting in blistering at the basal layer. Other keratin pairs are involved in a variety of diseases of epidermis, hair, and nails.

Wound Treatment

The primary goal in the treatment of wounds is to achieve wound closure for which several modalities have been developed. One of the persistent problems in the treatment of large wounds, like burns, is the availability of skin cover to repair damaged areas.

The treatment adopted depends on the type of wound and the physician's approach. Post-operative wounds are typically categorized into primary intention (surgical closure), secondary intention (wound left open to close by reparative process) and tertiary closure (left open, usually because of infection, and closed surgically at a later date). In primary intention the physician approximates the wound edges resulting in minimal scar formation.

Secondary intention wound treatment usually occurs when there are, for example, (a) gaping wounds with lacerated edges, (b) large defects that cannot be covered by grafting, (c) extensive trophic disturbances such as leg ulcers, (d) highly suppurative wounds, (e) wounds interspersed with foreign bodies, (f) infected wounds that have undergone primary closure, and (g) wounds that heal better cosmetically and functionally as a result of contraction rather than sutures, e.g. fingertip injuries. With secondary intention wound treatment, the loss of tissue has to be compensated by the formation of new connective tissue and wound contraction. Scar formation under such conditions can often result in a cosmetically and functionally inferior scar.

In these situations, sealing of epithelium across the wound does not occur rapidly, as the cells have to grow down and spread progressively across the wound at the junction of viable and non-viable tissue. There is also more granulation tissue formation growing from the base of the wound to fill the defect. This is accompanied by wound contraction, causing the end result to be an intact epithelium. However, more tissue distortion and an extensive, cosmetically unsatisfactory, scar often causes an impairment of function. Goepel JR. “Responses to cellular injury,” In: Underwood JCE (ed). GENERAL AND SYSTEMIC PATHOLOGY, Second Edition. London, UK, Churchill Livingstone, 1996, pp 121-122.)

In mammalian skin, this type of tissue injury initiates a complex but orderly series of biochemical and cellular events that are influenced by a large number of chemical mediators, leading to haemostasis, wound healing and the eventual generation of the scar tissue. The repair process can be arbitrarily divided into three main overlapping and inter-relating phases: inflammation; new tissue formation (proliferation); and matrix formation and remodeling. (Clark R A F. Wound repair; overview and general considerations. In: Clark R A F (ed). THE MOLECULAR AND CELLULAR BIOLOGY OF WOUND REPAIR, Second Edition. London, UK, Plenum Press, 1996, pp 3-50)). It should be emphasized that there is no clear demarcation between the phases of wound healing, as the process of tissue repair is a continuous phenomenon.

Wound healing of the skin is thus a complex biological process that requires the restoration of cover by re-epithelialization and restoration of support by dermal fibroblasts. Re-epithelialization results from keratinocyte migration and proliferation. The rate of re-epithelialization is higher in superficial wounds as compared to deeper wounds wherein the chances of infection and scar formation are very high. While the primary end point of healing is the wound closure, the focus on the quality of healing is also important.

Tissue-engineered skin substitutes are a significant advance in the field of wound healing. These were developed due to limitations associated with the use of autografts, including the creation of a donor site, which is at risk of developing pain, scarring, infection and/or slow healing, for example.

Prior to the development of tissue-engineered skin, the only available options were split or full-thickness skin grafts, tissue flaps or free-tissue transfers. Over the last two decades tissue-engineered skin substitutes have been developed and their use has progressed at a very rapid rate. Tissue engineering was defined in 1987 by the National Science Foundation bioengineering panel meeting in Washington, D.C., USA, as “the application of the principles and methods of engineering and the life sciences toward the development of biological substitutes to restore, maintain, or improve function.” Today, tissue engineered skin products have been approved for use by the US Food and Drug Administration (FDA) and others are undergoing testing and regulatory review. Tissue-engineered skin substitutes offer the promise of tissue replacement without requiring a donor site and may produce better healing.

Tissue-engineered skin may function by providing at the wound site the needed matrix materials or cells required for the healing process. Tissue-engineered skin refers to skin products made mainly of cells, or extracellular matrix materials only, or to a combination of cells and matrices.

Cell sources for tissue engineering fall into three categories: autologous cells (from the patient); allogeneic cells (from a human donor, but not immunologically identical); and xenogeneic cells (donor from a different species). Each of these origins may further be delineated into adult or embryonic stem cells, or “differentiated” cells obtained from tissue, where the cell population comprises of mixture of differently matured cells that includes rare stem and progenitor cells. To date, the available therapies depend on the whole cell mix, or separation of enrichment of stem cells.

While autologous tissue transfers can be highly effective in securing wound healing, such procedures (e.g., grafts and flaps) are invasive, painful, and expensive, and are not within the purview of many wound care practitioners. Autologous skin grafts, though successful, have limitations due to the limited donor sites as well as creation of fresh wounds at the donor site. To overcome this, several types of skin substitutes such as allogenic or xenogenic skin grafts have been used with varying degrees of success. Limitations of this treatment is related to sterility, difficulty in handling, risk of viral transmission and immune rejection by the host.

Cell-based wound therapies, such as cellular skin substitutes, have the potential to reduce wound contraction and to influence the nature of the final healed tissue. Reports are available that indicate additional benefits of skin substitute therapy besides early wound closure.

The property of the healed wound that closely resembles those of normal uninjured skin. Gohari et al., “Evaluation of tissue-engineered skin (human skin substitute) and secondary intention healing in the treatment of full thickness wounds after Mohs micrographic or excisional surgery,” Dermatol Surg 28:1107-14, 2002.)

It is believed that cells applied to the wound surface are “smart” and will bathe the wound bed with balanced cocktails of such mediators appropriate for the particular physiology of the wound environment that is sensed. Cells are thus advantageous over therapy provided by exogenous application of growth factors. Krishnamoorthy et al. “Specific growth factors and the healing of chronic wounds,” J Wound Care 10:173-8, 2001. For example, incubation of cells derived from biopsies of venous ulcers in conditioned medium supernatant from human fibroblast cell culture induces a highly significant increase in skin cell proliferation, and this effect has been correlated with levels of several cytokines. Martin et al. “Effect of human fibroblast-derived dermis on expansion of tissue from venous leg ulcers,” Wound Repair Regen 11:292-6, 2003.

A bio-engineered skin substitute has been to shown to act as an interactive “drug” delivery system. Shen et al. “Innovative therapies in wound healing,” J Cutan Med Surg 2003 (7(1) 217-224. Werner et al. and Mansbridge et al. have reported that the viability and metabolic activity of the cellular component of a cellular skin substitute is essential for therapeutic efficacy. These groups have proposed that this is due to the need for ongoing cytokine expression in the wound bed following application. Werner et al. “Regulation of wound healing by growth factors and cytokines,” Physiol Rev 83:835-70, 2003; Mansbridge et al. “Three-dimensional fibroblast culture implant for the treatment of diabetic foot ulcers: metabolic activity and therapeutic range,” Tissue Engineering 4:403-14, 1998. Measurements of matrix metalloproteinases (MMPs) secreted by the epidermis and dermis of the bioengineered skin substitute show that the individual layers of bio-engineered skin sense their environment and secrete MMP's in different quantities and ratios depending upon their environment. Osborne et al. “Epidermal-dermal interactions regulate gelatinase activity in Apligraf, a tissue-engineered human skin equivalent,” Br J Dermatol 146:26-31, 2002. Apligraf™ (U.S. Pat. No. 4,485,096) is made from human fibroblasts grown on a semipermeable membrane with bovine type I collagen, then overlayed with keratinocytes which are grown until a confluent layer forms. The keratinocytes and dermal fibroblasts are derived from neonatal foreskin and propagated in culture. Subsequently, keratinocytes overlying the epidermis are exposed to an air-liquid interface to promote formation of a stratum corneum. The process takes 20 days to be ready for application to the wound.

Epidermal substitutes prepared from keratinocytes obtained from a skin sample of the future recipient (autografts) or from an unrelated recipient (allografts) have been shown to have the ability to reconstitute an epidermis when delivered to an area of skin loss such as the excised bed of a burn eschar. (O'Conner et al., “Grafting of burns with cultured epithelium prepared from autologous epidermal cells,” Lancet I:75-78, 1981; Madden et al., “Grafting of cultured allogeneic epidermis on second- and third-degree burn wounds on 26 patients,” J Trauma 26(11):955-62, 1986).

Following the pioneering work of Rheinwald and Green, “Serial cultivation of strains of human epidermal keratinocytes: the formation of keratinizing colonies from cells,” Cell 6:331-344, 1975, it is now possible to serially propagate normal human keratinocytes in vitro. These keratinocytes can reconstitute a stratified squamous epithelium, which maintains the biochemical, morphological and functional characteristics of a native epidermis. Compton et al. “Skin regenerated from cultured epithelial autografts on full-thickness burn wounds from 6 days to 5 years after grafting. A light, electron microscopic and immunohistochemical study,” Lab. Invest 60:600-612, 1989. Established methods for in vitro culturing of epithelial grafts necessitate the growth of multi-layered cells. This procedure requires a period of 3-4 weeks before the films are ready for transplantation. The grafting of such labor-intensive sheets has faced mixed fortunes, however, with reports of lower than anticipated efficiencies. Desai et al. “Lack of long-term durability of cultured keratinocyte burn-wound coverage: a case report,” J Burn Care Rehabil. 12(6):540-5, 1991. Problems with the lack of “take” and long-term durability, time required for film preparation and high cost to produce such grafts, as well as difficulty in handling have led to the development of alternate delivery systems to transfer keratinocytes to the wound bed.

Cultured Epidermal Autografts (Epicel™) have been described as an in vitro cultured epidermal keratinocytes using a feeder layer of irradiated murine fibroblasts. A minimum of 2 to 3 weeks are required, from the time of biopsy collection to delivery of grafts Though these cultured epidermal autografts provides permanent wound coverage, a decreased requirement for donor sites, rapid coverage of the wound, faster pain relief, and a better functional and cosmetic outcome. However, the limitations of this graft include a 3-week delay for graft cultivation, the lack of a dermal component, and high cost.

Cultured allografts can be made in a similar process as Epicel™, derived from unrelated allogeneic donors, such as newborn foreskin. Since the cells can be grown in advance and stockpiled, the cells are readily available for grafting. For a number of technical reasons, this technology is not commercially viable.

The advantage of using cultured epidermal allografts over autografts is that allografts offer immediate graft availability, which can be important in treatment of severe injuries. The disadvantages of allografts are that they do not survive permanently on the wound bed, and there is a possibility of disease transmission, although, as with blood transfusions, this risk can be minimized with extensive screening.

An in vitro dermal replacement, Integra® is an artificial skin, which consists of an artificial dermis (matrix of bovine collagen and chondroitin-6-sulfate, a shark-derived glycosaminoglycan) and a disposable silicone sheet (artificial epidermis). It is approved for its use in burns. The advantage of using this artificial dermis is that it allows a neo-dermis to develop. Yet the disposable silicone sheet can allow the accumulation of exudate, increasing the risk of infection. It also does not provide a real epidermal component; and the silicone sheet must be surgically removed and ultimately replaced with an autograft or allograft.

Dermagraft™, a tissue engineered skin substitute, comprises collagen and glycosaminoglycans as a substrate for autologous cultured keratinocytes as the epidermal component, and collagen and glycosaminoglycan substrate inoculated with autologous fibroblasts as the dermal component. The overall ‘take’ was approximately 50%, however, attributed in part to proteases in the wound environment. This is not successful enough to make Dermagraft™ a routinely acceptable skin replacement. The additional disadvantage of this skin substitute is the need to wait 3 to 4 weeks to produce the cultured grafts.

Another skin substitute, previously available as Transcyte™, comprises neonatal (allogeneic) fibroblasts which have been cultured and proliferate on nylon fibers embedded into a silastic coated with porcine collagen. After 4 to 6 weeks this forms a dense cellular ‘tissue’, which contains high levels of secreted human matrix proteins as well as multiple growth factors. The fibroblasts are rendered nonviable by freezing after synthesizing collagen extracellular matrix and growth factors.

U.S. Pat. No. 6,790,455 describes a biodegradable and/or bioabsorbable matrix formed by electrospinning fibers of biodegradable and/or bioabsorbable fiberizable material physically associated with viable cells to contain and release the cells at a controlled rate.

U.S. Pat. No. 6,933,326 describes the Nonliving Allogeneic Acellular Dermal Matrix (marketed as Alloderm®), which is manufactured by cutting sheets of dry tissue matrix into strips; cryofracturing the dry tissue matrix strips at cryogenic temperatures; and freeze drying the strips to remove any moisture that may have been absorbed to give a dry acellular tissue matrix.

U.S. Pat. No. 4,963,489 describes living stromal tissue prepared in vitro, comprising stromal cells and connective tissue proteins naturally secreted by the stromal cells attached to and substantially enveloping a framework composed of a biocompatible, non-living material formed into a three dimensional structure with interstitial spaces. The related commercial product marketed as Dermagraft® is produced by culturing human dermal fibroblasts onto a biosynthetic scaffold. As the fibroblasts proliferate on the scaffold, they secrete important structural proteins and growth factors, generating a three-dimensional human dermis. Dermagraft is then frozen for storage and shipment to the treating physicians for implantation into patients.

U.S. Pat. No. 6,638,709 describes a composite cultured skin, which consists of allogeneic fibroblasts and keratinocytes grown in vitro and seeded on opposite sides of a bilayered matrix of bovine collagen, and marketed as OrCel™.

Although there is very little information about biological materials derived from pigs being used as wound dressings they are thought to act as dermal matrices. It has been reported that the structure and biochemical composition of small intestinal mucosa supports tissue-specific remodeling.

U.S. Pat. No. 5,882,521 describes a product derived from porcine small intestinal submucosa which is commercially available as Oasis™ wound dressing. Another biosynthetic, acellular, porcine derived dermal matrix is E-Z-Derm™. It is available as a perforated or nonperforated dressing attached to a gauze liner that is discarded before application. The advantages of acellular, porcine derived matrices are immediate availability for serial grafting until autografts are available, and long shelf life. The disadvantages include the possibility of disease transmission and poor drainage from the wound, increasing the accumulation of exudate and resultant risk of infection. There is a scarcity of clinical data supporting the tolerability and effectiveness of these types of dermal matrices.

In severely burnt patients, early wound excision and skin grafting plays a crucial role and has resulted in shortened hospitalization time, decreased complications, and increased survival. Conventional treatment of deep or full-thickness burn wounds is achieved by using split-thickness skin grafts (STSG) as wound coverage. However, disadvantages associated with this method include that it causes morbidity at the donor site and that there are limited donor sites in extensive burns.

Several methods have been used for delivering highly proliferative keratinocytes. One way in which cells can be made available at the wound site is through the culture of keratinocytes on the surface of biodegradable membranes which, when placed on the wound would release the cells on breakdown. Ronfard et al., “Use of human keratinocytes cultured on fibrin glue in the treatment of burn wounds,” Burns 17(3):181-4, 1991.

The second method involves the “upside-down” transfer of cells cultured on non-biodegradable membranes. Wright K A et al. “Alternative delivery of keratinocytes using a polyurethane membrane and the implications for its use in the treatment of full-thickness burn injury,” Burns 24(1):7-17, 1998. Studies performed on animals demonstrate the ability of keratinocytes to migrate from the membrane onto the wound bed and reconstitute an intact epidermis. Alternatively, a suspension of keratinocytes could be administered at the wound site after incorporating into fibrin glue. Kaiser et al. “Cultured autologous keratinocytes in fibrin glue suspension, exclusively and combined with STS-allograft (preliminary clinical and histological report of a new technique),” Burns 20(1):23-9, 1994.

### SUMMARY OF THE INVENTION

The present invention provides a technique of culturing human epidermal cells into a proliferating, sub-confluent layer on a biocompatible membrane, to form sheets suitable for grafting. The present invention also provides a reconstructive procedure to meet the specific requirements necessary to achieve satisfactory wound closure and also to restore functional integrity in the least time and with the least complications and morbidity.

In one embodiment, the present invention provides an interactive wound cover comprising an epidermal component. In particular embodiments, the wound cover comprises a preconfluent (or sub-confluent) monolayer of cultured, actively proliferating epidermal epithelial cells on a biocompatible polymer. For example, in some embodiments the wound cover comprises a biocompatible polymer, such as PLA, PGA and the like, onto or into which are cultured epithelial cells from the epidermis. Such epithelial cells include keratinocyte stem cells, basal keratinocytes, keratinocyte transient amplifying cells, proliferating keratinocytes, for example. In one embodiment, the cells are undifferentiated proliferating keratinocytes (basal keratinocytes).

In related embodiments at least 90% of said cells are actively proliferating keratinocytes. The epithelial on the biocompatible polymer are, in some embodiments, preconfluent, including less than 20%, less than 50%, less than 75%, less than 80%, less than 90%, less than 95%, and less than 99% confluent. The present compositions possess a number of advantages over other compositions in the art, including that it (a) adheres quickly to the wound; (b) mimics the physiology and some of the mechanics of the normal skin; (c) is highly effective in accelerating tissue regeneration and wound repair; and (d) can be made available in less time with a commercially viable process. The present wound treatment compositions are also effective and safe. An additional advantage of the present invention is that, in some embodiments, the keratinocytes retain substantial viability on the wound cover for at least 72 hours after application to a wound. Substantial viability is 75-90% viability.

In related embodiments, the present invention provides for methods and processes of preparing a wound cover. In one embodiment, a process for the preparation of a wound cover comprises the steps of: (a) isolating epidermal cells from the donor skin; (b) preparing a suspension of proliferating basal cells (c) expanding the cells (d) cryopreserving the cells and thawing when needed; (e) seeding cells onto PLA sheets in a transport device comprising transport media. In one embodiment, keratinocytes are seeded onto PLA sheets in at a concentration of 0.10×10⁶ to 0.5×10⁶ cells/cm.² In related embodiments, the wound cover is transferred to a transport device made of polycarbonate. The transport media may comprises carbon dioxide enriched media. In embodiments where the donor skin is autologous, for the construction of an autologous skin graft, cryopreservation may be used. When the donor skin is allogenic, cryopreservation is more common, so that a bank of cryopreserved cells can be available for rapid production of wound covers, as needed. In further embodiments, the invention comprises a kit for the preparation of wound covers. Such a kit includes, in one embodiment, (a) sterile sheets of biocompatible polymers; (b) a device for growth and subsequent transport of the wound preparation. The kit may also include appropriate media, instructions, and may even include stocks of frozen keratinocytes which have been pre-screened to be suitable for forming the wound preparation in that they are viable and free from pathogens and other contaminants.

The presently inventive composition find use in a variety of therapeutic modalities, such as a tissue substitute. It addresses the problem of severe of chronic wounds by accelerating skin tissue regeneration and wound healing by stimulating the recipient's own wound bed-derived skin cells. This includes wounds such as a post-operative wound, a burn, a diabetic ulcer, a bed sore, and a traumatic injury. The present invention is suitable for use in a variety of animals, including domesticated and agricultural animals, in veterinary practice, or on human patients.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1: Demonstrates that cells obtained from the skin biopsies retain viability following storage at various temperatures.

FIG. 2A: Shows a wound cover of the present invention comprising cultured keratinocytes on polylactic acid (PLA) sheets.

FIG. 2B. Shows data of cell growth on tissue culture dishes and PLA films.

FIG. 3 Shows means of transport of the wound cover following growth.

A and B: Illustrate the transport container. A. Side view; B. Top view. The design of the transport container is registered under Indian Patent Application No. 195065, and is entirely incorporated by reference herein.

FIG. 3C: Shows cell viability during transport.

FIG. 3D: Shows data indicating the temperatures maintained by the insulated box during transport conditions.

FIG. 4: Mycoplasma testing of the cells as shown. 4A: Positive control; 4B: Cultured keratinocytes showing absence of mycoplasma.

FIG. 5: Shows results of cell identity testing by PAN-keratin antibody and MEL-5 antibody staining.

5A: Shows wound cover staining with PAN-keratin antibody. Keratinocytes are identified as fluorescent cells. Nuclei of cells are stained with DAPI.

5B: Shows wound cover staining with MEL-5 antibody. Melanocytes are visualized as green-yellow cells (visible in black and white by the shape of the cell) and cell nuclei are stained with DAPI, which identifies both melanocytes and keratinocytes. The figure demonstrates that melanocytes are a very small percentage of the cell mixture.

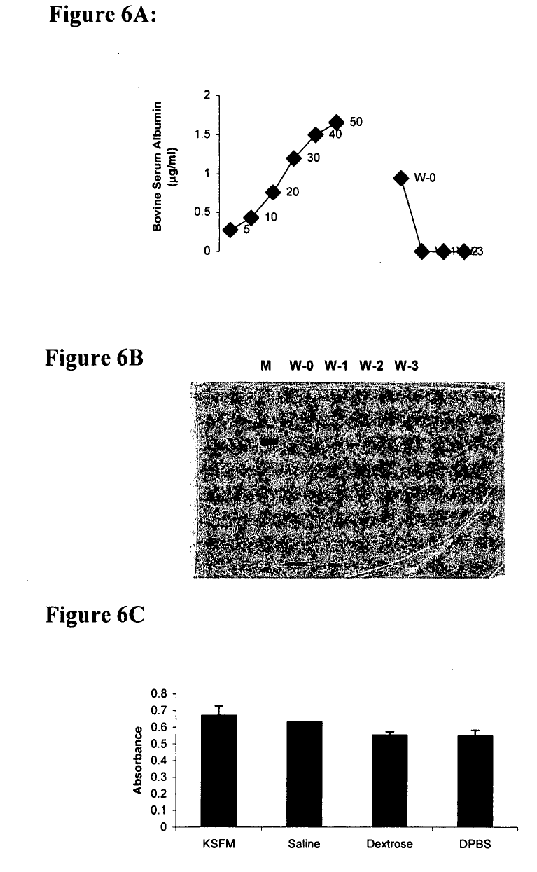

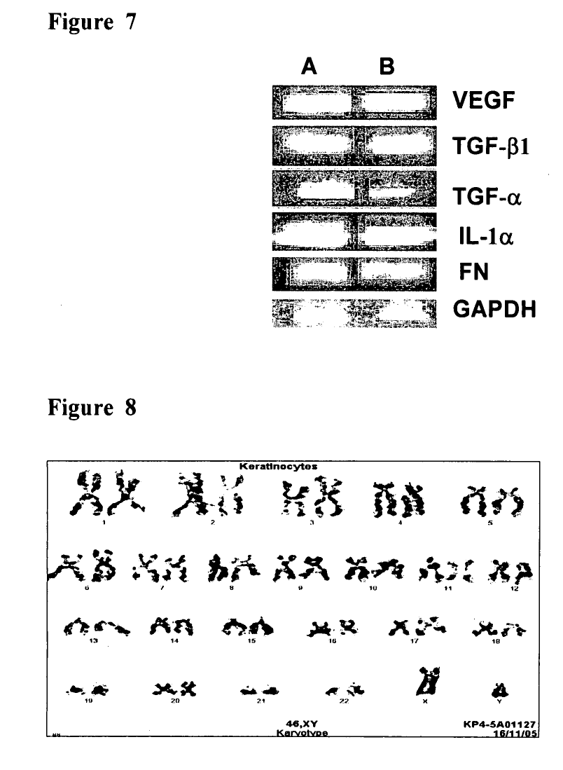

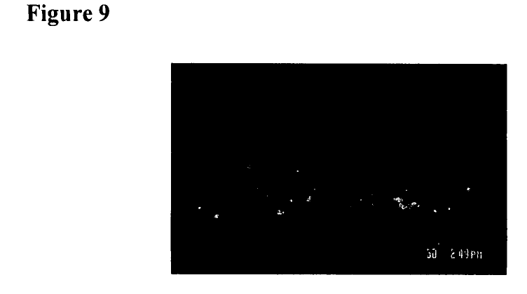

Under phase constrast microscope (20×) the keratinocytes may be seen as cuboidal as compared to melanocytes which have dendritic processes. The keratinocytes can be demarcated well from the melanocytes under an inverted microscope by their morphology. This can also be seen in the comparison of FIG. 5A (showing keratinocytes) and 5B (which show a melanocyte and the nuclei of many keratinocytes).

FIG. 6: Determination of removal of reagents from the final product at the time of application.

FIG. 6A: Demonstrates that rinsing removes residual proteins from the wound cover.

FIG. 6B: SDS-PAGE analyses of the residual proteins in the rinse. Legend: M: Mol. wt. marker; W-0: Transport media; W-1: 1st rinse; W-2: 2nd rinse; W-3: 3rd rinse.

FIG. 6C: Demonstrates that rinsing of graft before transplantation does not effect cell viability. Cell viability was determined with the MTT assay, measured with absorbance. KSFN and isotonic rinsing solutions did not affect cell viability.

FIG. 7: Expression pattern is identical between keratinocytes cultures on tissue culture plastic and those cultured on PLA. A: Keratinocytes cultured on tissue culture plastic; B. Keratinocytes cultured on PLA.

FIG. 8: Demonstrates that cultured keratinocytes show a normal karyotype.

FIG. 9: Presence of fluorescent labeled cells in the wound bed indicate migration of keratinocytes from PLA to the wound bed.

### DETAILED DESCRIPTION OF THE INVENTION

The present invention uses proliferative/preconfluent keratinocytes, whereby cells are transferred from culture to the wound bed before they form a sheet. This approach is advantageous over the prior art in that:
1. It aids in wound healing.
2. It ensures rapid coverage of wound.
3. It aids in relieving pain by adhering to the wound bed thereby sealing the nerve endings.
4. It ensures moist wound environment by preventing wound desiccation.
5. The grafts can be made within 3-4 days.
6. It is economical and offers an alternative treatment to the standard wound management therapies.
7. There is a dramatically reduced risk of transmission of infectious disease due to rigorous process controls.

The cells are grown directly on the delivery system such as a biocompatible polymer or biopolymer membrane. The cells can therefore be transferred as such to the patient thus avoiding the potential damage occurring in the conventional enzymatic separation from the culture vessel. The cells are transferred while in a proliferative state. In some embodiments, the use of preconfluent cells aids in the adherence of such cells to the wound as they express a integrin profile different from fully differentiated, terminal keratinocytes.

The interactive component of the invention is provided by the use of actively proliferating keratinocytes. During tissue repair a number of cytokines, growth factors etc. are released at the wound site. The cells at the wound site express molecules that have both an autocrine as well as a paracrine effect. The expression of these factors however depends on the stage of wound healing. Skin cells administered at the wound site would, depending on the cues present at the wound site, either upregulate/downregulate certain factors and thus help in wound repair. Thus, the cells enhance healing by interacting with the factors present at the wound site.

The use of an interactive wound cover are useful for both repair and regeneration. Repair indicates the process that a tissue undergoes to completely regenerate/reform. Allogeneic keratinocytes used in this wound cover to repair the damaged skin leading to its regeneration.

In another embodiment, the process involves the optimization of scaffolds onto which cells are seeded to form a uniform tissue with scaffolds that provide physical and chemical cues to guide the process. Scaffolds may be selected from a group comprising of natural materials such as collagen and fibrin or synthetic materials such as degradable polyesters used in surgical sutures. Scaffolds take forms ranging from sponge-like sheets and fabrics to gels to highly complex structures with intricate pores and channels made with new materials processing technologies. The spatial and compositional properties of the scaffold, the porosity of the scaffold and interconnectivity of the pores are all required to enable cell penetration into the structure as well as the transport of nutrients and waste products.

To transfer preconfluent keratinocytes to a wound, a delivery system is required. Various methods have been described. Cells can be grown in a culture vessel, trypsinized, and applied directly in suspension or grown directly onto a delivery membrane that is then removed from the dish, inverted, and applied to the wound bed. In one embodiment, keratinocytes are cultured directly onto a delivery membrane (such as a biopolymer membrane) in a culture vessel, which is then peeled off when required for use. It is therefore possible to use keratinocytes before the cells achieve confluence. The membrane is then inverted and placed on the wound. This method eliminates the need for enzymatic release of cells before use, and permits the use of preconfluent, actively growing keratinocytes.

In another embodiment of the present invention, the wound cover include genetic modification of transplanted cells to improve wound healing transiently or to deliver gene products systemically.

Biocompatible polymers can be either natural or synthetic. In general, synthetic polymers offer greater advantages than natural materials in that they can be tailored to give a wider range of properties and more predictable lot-to-lot uniformity than can materials from natural sources. Synthetic polymers also represent a more reliable source of raw materials, one free from concerns of immunogenicity and pathogen contamination. Poly(lactic acid) (PLA), poly(glycolic acid) (PGA), and poly(lactic-co-glycolic acid) (PLGA) have also been called polylactide, polyglycolide, and poly(lactide-co-glycolide), respectively, according to the nomenclature system based on the source of the polymer. They have the advantage of not requiring surgical removal after they serve their intended purposes. PGA, PLA, and especially their copolymers PLGA are the most commonly used family of biodegradable polymers. PGA was used as a biodegradable suture material in the 1970s, and it has led the largest volume production in the biomedical polymer markets, when its production was combined with those of PLA and PLGA. These polymers have found a broad range of pharmaceutical and biomedical applications based on their unique properties, including versatile degradation kinetics, non-toxicity, and biocompatibility. PGA is a highly crystalline polymer and the most hydrophilic among them. It has a very high melting point (224° C. to 226° C.), and the degradation rate of PGA is much higher than that of PLA. Random PLGA copolymers with different ratios of lactide (LA) and glycolide (GA) exhibit different degradation rates, and thus can be tailor-made for specific applications requiring specific degradation kinetics ranging from weeks to months. They are generally more amorphous than their homo-polymers and become most susceptible to hydrolysis when the two-monomer contents are the same. PLA and PGA are among the few synthetic degradable polymers that have been approved for clinical use and these have been widely studied in tissue development. PLA or polylactide is prepared from the cyclic diester of lactic acid (lactide) by ring opening polymerization. Lactic acid exist as two optical isomers or enantiomers. The L-enantiomer occurs in nature as D.L-racemic mixture results from the synthetic preparation of lactic acid. Fibers spun from “L” polylactide (melting point 170° C.) have high crystallinity when drawn whereas fibers spun from poly DL-lactide are amorphous. Crystalline poly-L-lactide are more resistant to hydrolytic degradation than the amorphous DL-form. The factors affecting the mechanical performance of biodegradable polymers are well known to the polymer scientist, and include monomer selection, initiator selection, process conditions, and the presence of additives. These factors in turn influence the polymer's hydrophilicity, crystallinity, melt and glass-transition temperatures, molecular weight, molecular-weight distribution, end groups, sequence distribution (random versus blocky), and presence of residual monomer or additives. In addition, the polymer scientist working with biodegradable materials can evaluate each of these variables for its effect on biodegradation.

Although the characteristics of these polymers are well understood, they merely serve to provide a 3-D biocompatible structure onto which cells can attach and do not interact with the cells. In the body, cells are situated within an extracellular matrix (ECM), which provides tissues with the appropriate architecture as well as signaling pathways that influence key cell function such as migration, proliferation, and differentiation. Research continues on the utilization of matrix molecules along with specific growth factors to optimize cell adherence to the scaffolds and direct cell activity.

Culturing sub-confluent keratinocytes on a delivery system avoids some of the disadvantages facing existing cultured sheets of keratinocytes. Delivery of sub-confluent keratinocytes to the wound bed leads to its migration and colonization of the wound bed, release of growth factors at the wound site thereby aiding in wound healing. The use of polymers on which the skin cells are cultured and transferred, also improves the ease of handling and transfer of the keratinocytes. Additional advantages of using a transparent polymer includes microscopic observation of cells during processing, as well as the visualization of the underlying healing wound after its application to the wound bed.

In one embodiment, the present disclosure provides the culture and transfer of human epidermal keratinocytes for the treatment of epidermal wounds. The present invention aims at using a biocompatible polymer. Polymer sheets selected from PLA and PGLA, for example, have the advantages of ease of handling during culture and application on the wound bed, and the ability to stick to the contours of the wound. PLA sheets are transparent, permitting visualization of the cells during culture as well as the healing process after its application on the wound site. Further it possesses barrier properties, preventing microbial contamination of the wound site.

The present invention provides the use of sheets made of biocompatible, biodegradable polymer such as PLA as a delivery system for keratinocytes as well as the ability of using this system as a tool to deliver proliferating keratinocytes to the wound bed.

In one embodiment, a wound cover of the present invention comprises cultured keratinocytes on PLA in a specially designed container. The crude PLA is prepared by catalytic reaction using dilactide and is purified by re-dissolving in a solvent like acetone, chloroform which is then re-precipitated with water. In this process the unconverted dilactide, other monomers, and impurities will be removed along with some portion of catalyst used. The polymer films are made by using purified PLA.

Besides eliminating the need for a second surgery, biodegradation of the biopolymer may offer other advantages. For example, a biopolymer layer may provide structural support and be engineered to degrade at a rate that will slowly transfer load to the healing tissue. Another exciting use for which biodegradable polymers offer tremendous potential is as the basis for drug delivery, either as a drug delivery system alone or in conjunction to functioning as a medical device.

The present disclosure provides biocompatible and biodegradable polymers for device designers and physicians to speed patient recovery and eliminate follow-up surgeries.

In one embodiment, raw materials used in the preparation of the wound cover of the present invention are sterile products purchased from companies such as Invitrogen and Sigma. Fetal bovine serum used for donor skin transport may be certified from BSE-free countries. Plastic-ware used in the manufacturing process may be disposable and obtained from NUNC™ (USA) and Falcon™ (USA). In another embodiment PLA membrane serves as a carrier of keratinocytes. PLA is cast into films and sterilized. The wound cover of the present invention may be transported in specially designed polycarbonate dishes. As part of the transport container for the wound cover of the present invention, silicone O-rings may be used. All raw materials may be tested to further ensure sterility of the materials. The process of manufacturing the wound cover of the present invention is carried out in clean rooms under cGMP norms.

In one embodiment, the wound cover of the present invention is for use in patients who have sustained partial thickness burns covering 10% or more of their total body surface area. Other possible uses include: donor site wound management, diabetic foot ulcers, venous ulcers, epidermolysis bullosa and cosmetic/aesthetic surgery.

The wound cover of the present invention may be ready for shipment in approximately 4 days after order from the clinician. The number of grafts required would be determined in advance based on the judgment of the physician, considering the patient's condition and the area of the wound to be covered. Each sheet may be applied to completely debrided, regenerating dermal wounds.

The wound cover of the present invention is available as a monolayer of epidermal cells cultured on approximately 16 sq. cm. circular sheet. The sheet with the cells facing upwards is supported inside a transport dish. Each graft is processed aseptically and packed individually under sterile conditions in transport media. The product is sealed in pouches specially designed and shipped in transport containers that maintain a temperature of 10° C.-25° C. for 72 hours.

The following examples are provided to demonstrate embodiments of the invention. Those of skill in the art will appreciate that the techniques disclosed in the examples represent techniques discovered by the inventors to function well in the practice of the invention, and thus are considered to constitute modes for its practice. However, those of skill in the art should, in light of the present disclosure, appreciate that changes may be made in the specific embodiments which are disclosed and still obtain a like or similar result without departing from the spirit and scope of the invention.

### EXAMPLE 1

### Preparation of the Wound Cover

A) Preparation of the PLA Sheets

The polymer was specially made by mixing equal amounts of L and DL dilactide, and polymerized to give PLA with a molecular weight of about 50,000-150,000 Da. A 20% (w/v) solution of the polymer was prepared in acetone and clarified to remove any suspended particles by centrifugation. The polymer film was then prepared by solvent casting method by pouring the solution over a solid support such as glass or steel plates. The sheets were allowed to dry by evaporation of solvent at ambient temperature; rinsed in sterile water and dried at ambient temperature. PLA film was sterilized with Ethylene Oxide (ETO).

Physical properties of ETO sterilized films were characterized using standard methods (IS/ASTM). Barrier property of the films against microbes (bacteria, yeast and fungi) was assessed by the ability of the films to prevent contamination of the underlying nutrient mix.

Physical characteristics of the films following ETO sterilization are presented in table 1.

TABLE 1

| | Properties | Results | Test |
|---|---|---|---|
| 1 | Appearance | Clear, | Visual and microscopic |
| | | transparent | |
| 2 | Thickness (μM) | 20-30 μ | Micrometer screw |
| 3 | Tensile strength | 400-500 μ | IS:2508 |
| | (Kg/cm²) | | |
| 4 | Modules of elasticity | 150-200 | ASTM-D-882 |
| | (Kg/mm²) | | |
| 5 | W.V.T.R (g/m²/24 hrs) | 250-300 | ASTM-E-398 |
| 6 | O.T.R (ml/m2/24 hrs) | 8000-12000 | ASTM-D-1434 |
| 7 | Barrier property | Bacteria and | Culture plates |
| | | Fungi | |

B) Collection of Donor Skin

Donor foreskin was collected from normal healthy neonates after receiving informed consent from the donor's guardian. The blood of the donor's mother was collected for testing to ensure the safety of donor skin cells to the recipient as well as the operator. The donor skin is taken for further processing only after the blood sample test for infectious diseases (I.D.) is negative for the following disease markers: HbsAg; Anti HIV 1 and 2; Anti HCV; CMV IgG; CMV IgM; and Syphilis IgG/IgM

C) Viability of Donor Biopsies

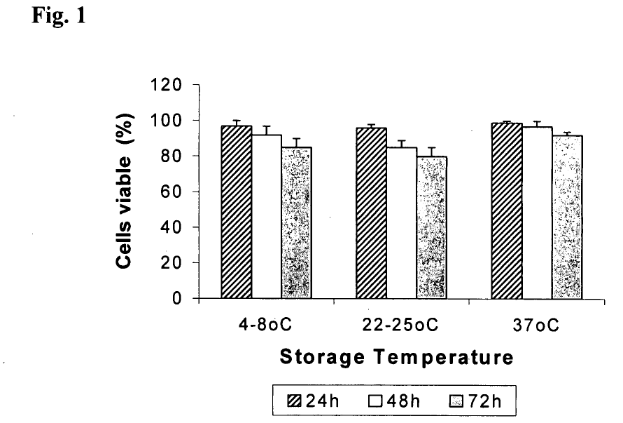

Skin samples were transported in specially designed insulated boxes (as described in Indian patent application 60/MUM/2006) that maintain a temperature of 5-25° C. for 72 h. To determine the effect of transport temperatures and its duration on the viability of cells obtained from the skin biopsy, freshly obtained skin was divided into three equal parts and stored at 4-8° C. (refrigerator); 22-25° C. (room temperature); and 37° C. (incubator). The skin was processed 24 h, 48 h and 72 h after storage. The epidermis was separated from the dermis, trypsinized, and the viability of the resulting single cell suspension was determined by the trypan blue dye exclusion method. Each experiment was performed with three individual donor skins. The data in FIG. 1 indicates that more than 80% cells were viable for 72 h under all transport conditions, with the maximum viability (92%) being observed in tissues that have been stored at 37° C.

D) Cell Culture Procedure

Cell Isolation and Expansion Involves the Following Steps:

1) The donor skin was trimmed, decontaminated, and epidermis was separated from the underlying dermis by enzymatic digestion.

2) A single cell suspension of the epidermis was made.

3) The viable cells were assessed and seeded in tissue culture dishes.

4) When the cells reach confluency, the cells were passaged. At the end of passage three or four, the cells were trypsinized and banked in a cryoprotectant solution.

E) Cell Growth on PLA Films

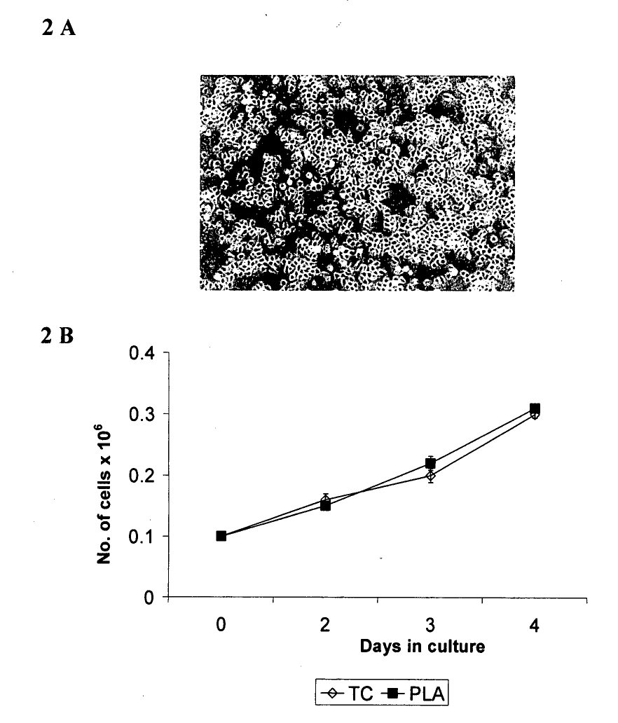

The ability of PLA films to support keratinocytes attachment and growth was determined by the cell growth assay. Briefly, keratinocytes (0.1×10⁶) were seeded on polystyrene dishes (TC dishes) (Nunc) or on PLA films placed in TC dishes and held in place with stainless steel rings. The cells were allowed to attach overnight and the media was replaced the next day (FIG. 2A). Keratinocytes cell growth was analyzed on days 2, 3 and 4 after seeding by trypan blue method following trypsinization. Each experiment was done in triplicate. The data presented in FIG. 2B shows no significant differences in cells numbers were observed between cells grown on TC dishes and PLA films. Accordingly, PLA films are an appropriate substrate for keratinocyte growth.

F) Culture and Transport of Cells on PLA Films

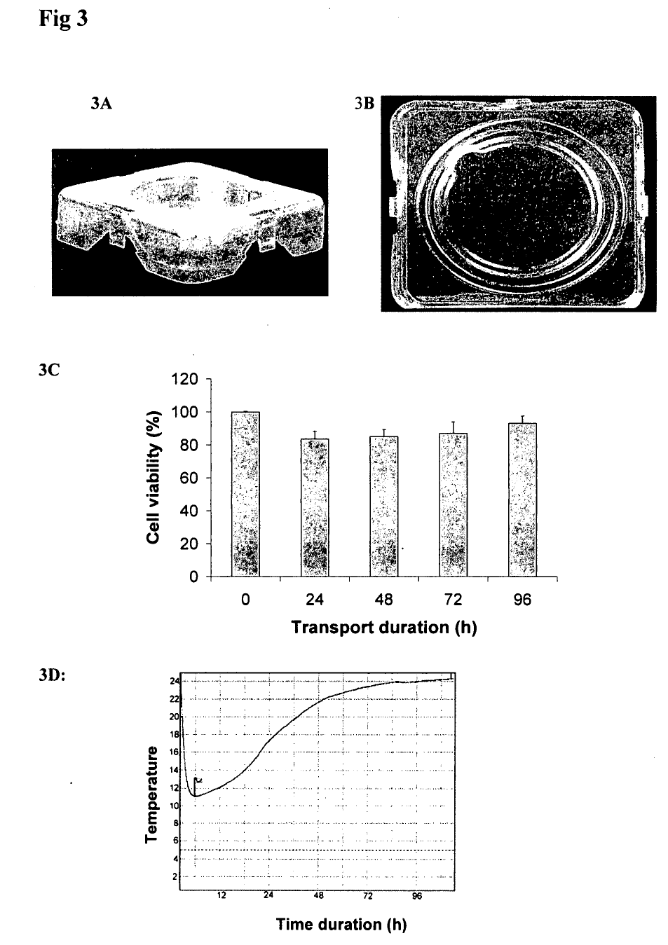

Sterilized PLA films were placed in specially designed transport dishes and soaked in PBS for 1 h. The film in each dish was held in place with polycarbonate ring. The cells were seeded at 3-5×10⁴ cells/cm² and cultured for 2 days before transport. For shipping, the media in the transport dishes was replaced with fresh media enriched with CO₂. The clasps on the dishes were closed securely thus ensuring an air tight seal. The sealed dishes were placed in insulated boxes for transport. Insulated boxes used for transport simulation studies were validated using Temp record data logger (Temp record International Ltd, USA). The design and the assembling of the transport container and its use in transport of cultured cells is elaborated in copending Indian patent application 60/MUM/2006, which is incorporated herein by reference. To retain the viability of cells during transport from a central processing centre to various tertiary hospitals, we used specially fabricated polycarbonate dishes (FIGS. 3A & 3B). These transparent dishes enable visualization of the cells during culture and are easy to handle during culture and transport.

To determine whether cells on PLA sheets retained their viability during transport, the dishes were subjected to conditions simulating transport, and the viability of the cells was assessed for four consecutive days by the MTT method. MTT [3-(4,5 dimethylthiazol-2-yl)-2,5-diphenyl tetrazolium bromide] assays were performed according to standard protocol of Hansen M B, Nielsen S E, Berg K., Re-examination and further development of a precise and rapid dye method for measuring cell growth and cell kill, J. Immunol. Methods 1989 119:203-210. Briefly, MTT (0.5 mg/ml) was added to the cells in culture media. At the end of 3 hours, the unreacted dye was removed, and isopropyl alcohol was added to the cells to dissolve the intracellular blue formazan crystals. The absorbance, determined as the difference in optical density measured at a test wavelength of 570 nm and a reference wavelength of 650 nm (Shimadzu UV-VIS Spectrophotometer, Japan) was compared to the absorbance obtained with cells attached on TC-dishes. The viability of the cells on the day of shipping was assumed to be 100%. Under transport conditions, about 80% of cells retained their viability for about 96 hours (FIG. 3C). It is assumed that, within 96 hours, the cells under shipping conditions could be transported to tertiary hospitals without much loss in viability. A slight increase in viability observed at 96 hours could be attributed to the temperature in the insulated box which had reached about 25° C. by 72 hours (FIG. 3D). Proliferation of some cells might have started at this point, leading to cell proliferation. The temperatures maintained by the insulated box during transport simulation condition are 11-24° C. for a period of 96 h (FIG. 3D).

### EXAMPLE 2

### Product Characterization and Testing

A) Microbial Testing:

1) Bioburden: The microbial contamination in the sample was determined in terms of numbers of colonies appearing on plates of solid media. The test involved the addition of sterile molten Casein Digest Agar to 1 ml of test sample in a petri dish. After solidification of the medium the plate was incubated at 30-35° C. in the inverted position for 48 hours. After this period the plates were visually inspected. No bacterial growth was observed.

2) Sterility testing: Sterility testing was performed to detect the presence of aerobic and anerobic microbes by inoculating the test samples in two different sterile nutritive medias namely Fluid Thioglycolate Medium (FTM) and Soybean Casein Digest Medium (SCDM). The result showed absence of growth during a period of 14 days for microorganisms such as bacteria, yeast and mold and for pathogens like E. coli, S. aureus, P. aeruginosa and Salmonella indicating sterility of the sample.

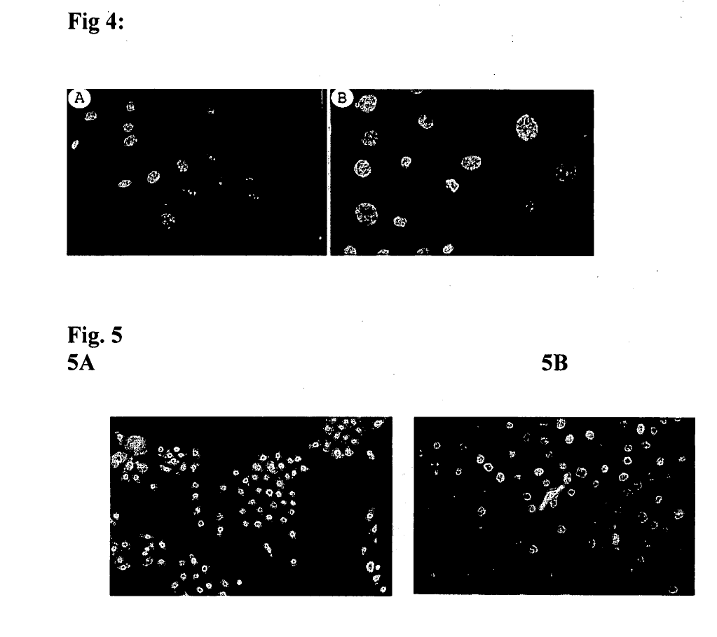

3) Mycoplasma testing: Mycoplasma contamination was assessed by a Hoechst staining kit. Following fixation and staining of the cells with Hoechst 33258, the cells were examined by fluorescence microscopy between 400-1000×. The positive cultures were identified by particulate or filamentous fluorescence around the cell nuclei, while negative cultures will only show nuclear staining as demonstrated in FIG. 4.

B) Cell Identification Testing

Cells were analyzed by immunohistochemical analyses using PAN keratin and MEL-5 antibodies. To determine the percentage of the keratinocytes in the cell at the time of cell banking, immunohistochemical analysis of the cells were performed. The identity of the cells was determined by immunostaining the cells with PAN-keratin antibody that specifically binds to keratinocytes. Presence of the contaminating melanocytes was determined by immunostaining the cells with MEL-S antibody. (FIGS. 5 A and B) Positive staining with PAN-keratin was observed in 98-100% cells. Less than 2% cells stain positive with MEL-5 antibody.

C) Purity Testing

Determination of Removal of Reagents from the Final Product at the Time of Device Application

Cultured cells are shipped to hospitals in keratinocyte serum free media (KSFM, commercial media available from Invitrogen) media enriched with CO₂. To ensure the cells during transplantation are free from media components, the clinicians are advised to rinse the cells with normal saline solution.

To determine the number of rinses required for removing traces of the protein components present in the transport media, the cells were rinsed with saline. At the end of each rinse, the wash solution was collected and the protein concentration estimated using a spectrophotometer (FIG. 6A). FIG. 6B shows the rinses that were analyzed on SDS-PAGE gels and stained with coomassie blue. FIG. 6C shows how that the viability of keratinocytes are not affected by washing keratinocytes with isotonic solutions like saline, dextrose, etc. Bovine serum albumin was used as standard. On analyses, it was seen that the concentration of proteins in the wash solution falls below detectable levels at the end of the first rinse. However, to ensure complete removal of all protein traces, the clinicians are advised to rinse the grafts at least three times before grafting. To determine the effect of rinsing keratinocytes with various isotonic solutions, the cell viability was assessed by the MTT method after washing the cells thrice with the wash solution followed by 48 h incubation. KSFM rinsed keratinocytes served as controls. Rinsing did not affect viability.

D) Potency Testing: Gene Expression.

Keratinocytes secrete growth factors and extracellular matrix proteins that aid in wound healing. Keratinocytes cultured on PLA retained the expression of growth factors similar to that of tissue culture dishes, indicating their capability to enhance wound repair. The expression of growth factors and extracellular matrix in PLA cultured keratinocytes vs. tissue culture dishes were assessed by RT-PCR. The genes analyzed were vascular endothelial growth factor (VEGF), transforming growth factor-alpha 1(TGFα1), transforming growth factor beta (TGF β), interleukin 1 alpha (IL-1α) and fibronectin (FN). Products of all these genes play an important role in wound healing. To ensure equal amounts of RNA from both the groups were used in the experiment, glyceraldehyde phosphate3-dehydrogenase (GAPDH), a house-keeping gene was used as an internal control. As seen in FIG. 7, no differences in expression patterns were observed in keratinocytes cultured on PLA as compared to those on tissue culture dishes.

E) Safety Testing

1) Tumorigenicity

To ensure that the cultured keratinocytes do not have any abnormalities/transformations that might lead to tumor formation in the recipients, tumorigenicity assays were conducted in vitro. The ability of the cultured cells to form colonies in soft agar was assessed as an indicator of possible transformation and the potential ability for the cells to form tumors in humans. Typically, a single-cell suspension of cultured cells is inoculated in a soft agar overlay of a hard agar base. Cultures were monitored for 28 days for the formation of colonies of greater than the cells. No colonies formed in cultures inoculated with the lots of cultured cells tested.

TABLE 3
Illustrates the results of the in vitro tumorigenesis assay indicating
the safety of the cultured keratinocytes for transplantation

| | | | % Colony |
|---|---|---|---|
| Cell type | No. of samples | No. Analyzed | formation |
| Keratinocytes | 7 | 7 | 0 |
| Positive control | 2 | 2 | 100 |
| melanoma | | | |
| Negative control, adult | 1 | 1 | 0 |
| fibroblasts | | | |
| Negative control, neonatal | 1 | 1 | 0 |
| fibroblasts | | | |

2) Karyology:

Karyological analysis was conducted on cultured keratinocytes to determine the number of chromosomes and check for the presence of abnormalities. Cells were swollen, fixed onto slides, and stained with Giemsa stain. Twenty metaphases were examined for chromosome count and three metaphases were karyotyped (FIG. 8). In the three lots analyzed, the results showed a normal 46,XY karyotype consistent with the patient's known sex. There was no evidence of clinically significant numerical or structural chromosome abnormalities.

F) Stability Testing

1) Stability of the Banked Cells:

The viability of the cryopreserved keratinocytes was determined for a period of 3 years. The parameters that were analyzed were a) cell viability b) cell attachment and c) cell morphology. The results are shown in the table below:

TABLE 4

| Period of | % Cell | Cell | Cell |
|---|---|---|---|
| analysis (months) | Viability | Attachment | Morphology |
| 6 | 95 ± 3 | Good | Cuboidal at >65-70% |
| | | | confluency |
| 12 | 95 ± 3 | Good | Cuboidal at >65-70% |
| | | | confluency |
| 18 | 95 ± 3 | Good | Cuboidal at >65-70% |
| | | | confluency |
| 24 | 95 ± 5 | Good | Cuboidal at >65-70% |
| | | | confluency |
| 32 | 95 ± 5 | Good | Cuboidal at >65-70% |
| | | | confluency |
| 36 | 95 ± 6 | Good | Cuboidal at >65-70% |
| | | | confluency |

The results indicate that keratinocytes recovered over a period of 3 years after freezing were stable for transplantation.

2) Stability of the Wound Cover:

The banked keratinocytes were thawed and seeded on PLA sheets in transport dishes. At the end of the culture period one set of dishes in triplicate was checked for viability by MTT assay. For the purpose of this study this data was assumed as 100% viable. Transport dishes were then stored at either 4-8° C., 22-25° C. and 37° C. These temperatures were selected to cover the entire range of temperatures that the final product would be exposed during shipment. During the experiment, the final product was placed in the refrigerator to simulate 4-8° C. transport conditions, room temperature (22-25° C.) and incubator (37° C.). The frequency of the assessment was after 24, 48 and 72 hours. The pH of the shipping media and viability of the cells were analyzed. Results are tabulated below:

TABLE 5
Data for Wound cover stored at 4-8° C.

| Parameters Analysed | 24 h | 48 h | 72 h |
|---|---|---|---|
| pH of the shipping media | 5.8 | 6.3 | 6.8 |
| % viable | 105 ± 10 | 110 ± 8 | 90 ± 12 |

TABLE 6
Data for wound cover stored at 22-25° C.

| Parameters Analysed | 24 h | 48 h | 72 h |
|---|---|---|---|
| pH of the shipping media | 6.0 | 6.8 | 7.4 |
| % viable | 121 ± 9 | 125 ± 12 | 90 ± 7 |

TABLE 7
Data for wound cover stored at 37° C.

| Parameters Analysed | 24 h | 48 h | 72 h |
|---|---|---|---|
| pH of the shipping media | 6.1 | 7.0 | 7.7 |
| % viable | 122 ± 10 | 186 ± 13 | 186 ± 15 |

The increase in viability seen at 22-25° C. and 37° C. is attributed to the proliferation of the keratinocytes that takes place at this temperature. Because the final shipping container, as disclosed herein, ensures that the temperature is retained between 5-25° C. for 78 hours and the device should be applied within 72 hours after shipping, stability of the product during shipping is ensured.

3) Stability of the Transport Conditions:

Because the wound cover of the present invention should be shipped in a specially designed transport container inside an insulated box, the ability of the insulation box to maintain shipping temperatures was assessed.

About 10 transport containers comprising the wound cover of the present invention were placed inside an inner insulated box. Temperatures of the wound cover located inside the inner insulated box was monitored using the temperature record logger and analyzed. Six data points per hour were monitored for a period of 78 hours. The data showed that temperatures were maintained between 5-25° C. for 78 hours.

### EXAMPLE 3

### In Vivo Testing

A) Migration of Keratinocytes from PLA

To enhance wound healing, keratinocytes should be delivered to the wound bed. Once the wound cover is applied to the wound bed, cells must migrate from the PLA membrane to the wound bed. To confirm this event, an in vivo experiment was performed on guinea pigs. Briefly, partial thickness punch wound biopsies were created on the posterior portion of the animals. Cells in a wound cover of the present invention were labeled with a red fluorescent dye (CyDiI, Molecular Probes, USA) and then applied on the wound bed. At the end of 3 days, the wound was excised and processed for histological analysis. As shown in FIG. 6, fluorescent-labeled cells were visualized in the wound bed, indicating migration of keratinocytes from the PLA sheet to the wound bed.

B) Preclinical Evaluation:

The toxicity testing was done with a PBS solution with the PBS sheet for 72 h at 37° C. (hereinafter “test substance”). All toxicity studies were conducted in accordance with the Good Laboratory Practices (GLP) principles as published by OECD in 1998.

1) Toxicity:

Acute intracutaneous toxicity studies were conducted using the contact solution in male and female New Zealand white rabbits. The test was performed by intracutaneous administration of 0.2 ml test substance (contact solution) at five sites on one side of each rabbit. Similarly 0.2 ml of distilled water (control) at five sites on the other side of each rabbit was injected. The appearance of each injection site was observed immediately after injection and at 24, 48 and 72 hours after injection. Individual animals were then observed daily for the signs of toxicity for 14 days. The tissue reaction for erythema, oedema was graded for each injection site and at each time interval. After 72 hours grading all erythema grades and oedema grades were totaled separately for each test substance and control. Each total was divided by 36 (6 animals×3 grading×2 grading categories) to determine the overall mean score for each test substance versus the control. The requirements of the test were met if the difference between the test and the control mean score is 1.0 or less.

No adverse clinical signs or mortality was observed rabbits treated with the contact solution.

2) Allergenicity/Hypersensitivity:

The skin sensitization potential of the PLA contact solution in Guinea pigs was conducted according to GLP. The test was conducted on thirty Hartely strain guinea pigs divided randomly into 2 groups. The control group comprised 5 males and 5 females and the treatment group comprised of 20 guinea pigs (10 males and 10 females). Based on the results of the pilot study, the contact solution undiluted was selected for intradermal injection during induction exposure on day 0. The contact solution was found to be a non-irritant in a pilot study, and a shaved test site of guinea pigs was painted with 0.5 ml of 10% sodium lauryl sulphate in Vaseline to augment the local skin irritation. The undiluted contact solution was selected for topical application during induction (on day 7) and challenge exposure (on day 21). The skin reactions of the guinea pigs were recorded post induction (intradermal injections/topical application) following the Draize Method (Draize J. H., Woodward, G., and Calvery, H. O. 1944: Methods for the study of irritation and toxicity od substances applied topically to the skin and mucous membranes. J. Pharmacol. Exp. Ther., 82: 377-390) and at 24 h, 48 h, post challenge treatment following Magnusson and Kligman grading scale (Magnusson, B. and Kligman, A. M. 1969: The Identification of Contact Allergens by Animal Assay. The Guinea Pig Maximization Test, The Journal of Investigative Dermatology 52(3). 268-277).

No erythema and no oedema was observed on day 1 in all the guinea pigs from the treatment groups following intradermal injection (day 0). Very slight erythema (11/20 guinea pigs) was observed on day 10 on the left flank in the treatment group guinea pigs following topical application on day 7. No skin reactions were observed in the guinea pigs from the control group.

Visual observation of skin following challenge exposure did not reveal positive skin response at 24 and 48 hour post patch removal in the guinea pigs belonging to the treated groups.

No clinical signs related to the treatment other than skin irritation were observed during the course of the study.

A sensitization of zero percent was observed at 24 and 48 hour post patch removal in the present study using an adjuvant.

3) Hemolysis Study:

The hemolysis study of the PLA contact solution was tested in rabbit blood. The study involved the effect of the four concentrations of the test substance on the heparinized blood of the healthy rabbit. The four concentration of the test substance 0.25, 0.5, 0.75, and 1 ml were taken and the test mixture without the test substance served as a negative control. Sodium bicarbonate was used as a positive control. The test involved addition of the test substance with 25 ml Saline buffer with pH 7.4 to 0.4 ml of heparinized blood. This mixture was incubated for 1 hour at 37° C. After incubation the mixture was centrifuged at 3500 rpm for 5 minutes and the supernatant was separated. The supernatant was read under UV absorbance at 545 nm. The percentages of hemolysis were 1.09, 1.64, 2.72, and 2.72 against 0.25, 0.5, 0.75 and 1 ml of the test substance. Hence from the study the maximum non-hemolytic dose was 1 ml.

4) Mutagenicity:

The mutagenicity of the contact solution was evaluated by the Ames Salmonella typhimurium—reverse mutation assay. The test was conducted in 5 tester strains of Salmonella typhimurium (TA 97a, TA98, TA 100, TA 102 and TA 1535).

The test substance dissolved in distilled water was tested at the doses of 5000, 500, 50, 5 and 0.5 ug/plate. Simultaneously cultures in control received distilled water or positive mutagens (benzo-a-pyrene, 2-nitrofluorene, sodium azide, 9-aminoacridine and Mitromycin C (obtained from Sigma/Aldrich, USA). In order to study the role of metabolic activation, cultures were incubated with and without S9 mixtures. The induction of Histidine positive colonies was computed and results were statistically treated for comparison.

The study indicated lack of statistically significant induction of His+ revertant colonies in any of the tester strains with or without S9 addition in the culture. The mean numbers of revertant colonies obtained with the plates treated with highest concentration of test substance (5 mg/plate) in the presence and absence of S9 mix were comparable to negative control plates. The positive mutagens used in this study resulted in a strongly positive mutagenic response by inducing a multi-fold increase in the number of His+ revertant colonies over the negative control.

5) Clinical Evaluation

The safety and efficacy of the interactive wound cover of the present invention was studied in human subjects and the timely wound closure of the split thickness donor sites was compared with a commonly used surgical dressing. The results showed increased wound healing with respect to epithelialization as compared to the controls.

All references mentioned or cited herein are entirely incorporated by reference herein as if included in the body of the present specification.

All of the compositions and methods disclosed and claimed herein can be made and executed without undue experimentation in light of the present disclosure. While the compositions and methods of this invention have been described in terms of certain embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition for therapeutic use as a wound cover, comprising (i) epithelial cells derived from allogenic or autologous epidermis and (ii) a biopolymer membrane polymer selected from the group consisting of polylactic acid (PLA), polyglycolic acid (PGA) and polylactic-polyglycolic acid copolymer (PLGA).

2. The composition of claim 1, wherein the epithelial cells are present as a monolayer on the biopolymer membrane.

3. The composition of claim 2, wherein said monolayer is a sub-confluent monolayer.

4. The composition of claim 3, wherein said monolayer is at least 20% confluent.

5. The composition of claim 4, wherein said monolayer is at least 50% confluent.

6. The composition of claim 1, wherein the epithelial cells are keratinocyte cells.

7. The composition of claim 6, wherein the keratinocyte cells are keratinocyte stem cells.

8. The composition of claim 1, wherein the epithelial cells are transient amplifying cells.

9. The composition of claim 6, wherein the keratinocyte cells are present on the biopolymer membrane in a sub-confluent monolayer.

10. The composition of claim 6, wherein the keratinocyte cells comprise cells capable of further proliferation.

11. The composition of claim 6, wherein the keratinocyte cells comprise undifferentiated cells.

12. The composition of claim 1, wherein at least 90% of the epithelial cells are actively proliferating keratinocytes.

13. The composition of claim 1, wherein said cells retain substantial viability on the wound cover for at least 72 hours after application to a wound.

14. The composition of claim 1 wherein said biopolymer membrane is selected from biodegradable, biocompatible natural or synthetic material.

15. The composition of claim 1 wherein the biopolymer membrane selected from the group consisting of polylactic acid (PLA), polyglycolic acid (PGA) and polylactic-polyglycolic acid copolymer (PLGA) has a molecular weight of at least 50,000 Da.

16. A process for preparing the composition of claim 1 comprising the steps of:
a) isolating keratinocyte cells from allogenic or autologous epidermis;
b) preparing a cell suspension comprising the isolated keratinocyte cells;
c) expanding the keratinocyte cells;
d) optionally, cryopreserving the keratinocyte cells;
e) optionally, thawing the keratinocyte cells;
f) seeding the keratinocyte cells onto a biopolymer membrane;
g) expanding the keratinocyte cells into a sub-confluent monolayer on the biopolymer membrane; and
h) transporting the biopolymer membrane using a transport device comprising transport media.

17. The process of claim 16, wherein the keratinocytes cells are seeded onto biopolymer membrane sheets comprising PLA, at a cell concentration of 0.10×10⁶ to 0.5×10⁶/cm.².

18. The process of claim 16, wherein the wound cover is transferred to a transport device made of polycarbonate.

19. The process of claim 16, wherein the transport media comprises carbon dioxide enriched media.

20. A method of treating a wound in an individual comprising application of the composition of claim 1 to said wound in the individual, whereby said application is effective for the treatment of such wound.

21. The method of claim 20, wherein said wound is selected from the group consisting of:
a) a post-operative wound;
b) a burn;
c) a diabetic ulcer;
d) a bed sore; and
e) a traumatic injury.

22. The method of claim 20, wherein said individual is human.

23. A composition comprising a wound cover comprising a sub-confluent monolayer of keratinocyte cells and a biopolymer membrane.

24. A composition comprising a wound cover comprising a monolayer of undifferentiated keratinocyte cells on a biopolymer membrane.

25. A wound cover composition comprising (1) a sub-confluent monolayer of keratinocyte cells comprising undifferentiated cells and (2) a biopolymer membrane, wherein the monolayer is on the biopolymer membrane.

26. A method for treating a wound in a subject, comprising using a biopolymer membrane as a delivery system, and delivering to the wound a sub-confluent monolayer of keratinocyte cells comprising undifferentiated cells.

27. A kit for treating a wound, wherein the kit comprises (1) a wound cover comprising (a) a sub-confluent monolayer of keratinocyte cells comprising undifferentiated cells and (b) a biopolymer membrane, wherein the monolayer is on the biopolymer membrane; and (2) a transport device.

28. A composition comprising a wound cover comprising a monolayer of proliferating basal epithelial cells on a biopolymer membrane.
